(19) United States
(12) Patent Application Publication
(10) Pub. No.: US 2010/0298280 A1
(43) Pub. Date: Nov. 25, 2010

(54) Compounds for the Modulation of Huntingtin Aggregation, Methods and Means for Identifying Such Compounds

(76) Inventors: Petra Kioschis-Schneider, Oftersheim (DE); Mathias Hafner, Mannheim (DE); Manuel Ammer-Schläger, Mannheim (DE); Sandra Ritz, Worms (DE); Andreas Holloschi, Mannheim (DE); Erich E. Wanker, Berlin (DE)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/602,200

(22) PCT Filed: Jun. 13, 2008
(86) PCT No.: PCT/EP2008/004797

§ 371 Date: Mar. 26, 2010

Related U.S. Application Data
(60) Provisional application No. 60/943,766, filed on Jun. 13, 2007.

(51) Int. Cl. A61K 31/58; A61K 31/352; C12N 15/12; C12N 15/63; C12N 5/10; C12Q 1/68; A61P 25/28

(52) U.S. Cl. 514/172; 514/453; 536/23.5; 536/23.4; 435/320.1; 435/325; 435/6

(57) ABSTRACT

The present invention relates to tetranortriterpenoid compounds and pharmaceutical compositions thereof, which are provided for use in the treatment, diagnosis and/or prevention of trinucleotide repeat disorders (like a polyglutamine diseases, e.g Huntingdon's disease), amyloid diseases, neurodegenerative disease, protein misfolding diseases or tumors. The tetranortriterpenoid compounds of the present invention are further provided for the reduction and/or inhibition of the aggregation of amyloidogenic proteins, preferably of polyglutamine proteins (such as huntingtin) as well as for increasing proteasome activity. The present invention furthermore relates to nucleic acids, comprising the nucleotide sequences of two huntingtin fragments, as well as to cells and kits, which are useful in methods for assessing the aggregation of huntingtin and in methods for identifying compounds, which modulate the aggregation of huntingtin.

The present invention relates to tetranortriterpenoid compounds and pharmaceutical compositions thereof, which are provided for use in the treatment, diagnosis and/or prevention of trinucleotide repeat disorders (like a polyglutamine diseases, e.g Huntington's disease), amyloid diseases, neurodegenerative disease, protein misfolding diseases or tumors. The tetranortriterpenoid compounds of the present invention are further provided for the reduction and/or inhibition of the aggregation of amyloidogenic proteins, preferably of polyglutamine proteins (such as huntingtin) as well as for increasing proteasome activity.

The present invention furthermore relates to nucleic acids, comprising the nucleotide sequences of two huntingtin fragments, as well as to cells and kits, which are useful in methods for assessing the aggregation of huntingtin and in methods for identifying compounds, which modulate the aggregation of huntingtin.

### BACKGROUND OF THE INVENTION

Aggregates of mutated proteins with amyloid structure are a hallmark of several neurodegenerative diseases like Alzheimer's Disease, Parkinson's Disease, amyotrophic lateral sklerosis and the polyglutamine (polyQ) diseases. In amyloid disorders mutated proteins show decreased solubility and accumulate in extra- or intracellular deposits by a mechanism that remains elusive (Lansbury & Lashuel, 2006).

Huntington's disease (HD) is a hereditary polyQ disease characterized by selective neuronal cell loss and astrocytosis mainly in the cerebral cortex and corpus striatum (Vonsattel 2007). Current drug therapy is limited to treat characteristic motor impairment with antichoreic/neuroleptic drugs, but there is no causative treatment to affect the progressive nature of the disease including dementia and psychiatric disturbances (Bonelli 2007.

HD is caused by an unstable CAG repeat expansion in the first exon of the huntingtin gene (IT-15) which translates into an elongated polyglutamine (polyQ) stretch in the protein huntingtin. A pathological polyQ length of more than 37 glutamine residues is associated with the appearance of cytosolic, perinuclear and nuclear inclusions containing aminoterminal huntingtin fragments and sequestered proteins e.g. ubiquitin, components of the proteasome, heat-shock proteins and transcription factors (Imarisio et al., 2008).

Since the discovery of huntingtin inclusions in postmortem brains of HD-patients and transgenic HD-mice (DiFiglia 1997), there is an ongoing discussion, if soluble mutated huntingtin, ordered intermediate structures (oligomers, protofibrilles, microaggregates) or large fibrilar aggregates are the primary toxic species (Arrasate 2004, Ross and Poirier, 2004).

Primary screening models for polyglutamine diseases suitable to screen large compound collections (10³-10⁶) focused currently on caspase-3 activity (Piccioni 2004), cytotoxicity (Igarashi 2003) and the aggregation of mutant huntingtin fragments (Zhang 2005). These assays were performed either in cell free systems (Wang 2005), yeast (Zhang 2005) or mammalian cells, respectively (Igarashi 2003, Pollit 2003).

Targeting the aggregation of mutant huntingtin in mammalian cells was aimed in a screening system based on the aggregation of fluorescent labelled mutant huntingtin fragments (HD17Q103-EGFP) in inducible PC12 cells (Apostol 2003, Bodner 2006). Pollit et al. (2003) developed an assay for the polyglutamine disease SBMA based on the transient expression of the androgen-receptor (ARQ112-EYFP, ARQ112-ECFP) in HEK293T cells using FRET between aggregated proteins as read out.

Recent evidence suggests that intermediates of the aggregation process like oligomers and protofibrils are likely to be the toxic species leading to neurodegeneration (Lansbury & Lashuel, 2006; Takahashi et al., 2008).

Therefore understanding the mechanisms of amyloid assembly and its impact in toxicity as well as modulating the aggregation process of huntingtin represents a promising strategy for a potential treatment of HD and an improved investigation of its role in pathogenesis.

Thus, the present invention aims to improve the methods and means of the art in the prevention, diagnosis and treatment of protein misfolding diseases like Huntington's disease (HD).

### SUMMARY OF THE INVENTION

According to the present invention this object is solved by providing tetranortriterpenoid compounds for use in the treatment, diagnosis and/or prevention of diseases, wherein the diseases are preferably a trinucleotide repeat disorders (like a polyglutamine diseases), amyloid diseases, neurodegenerative disease, protein misfolding diseases or a tumor.

According to the present invention this object is furthermore solved by providing tetranortriterpenoid compounds for use in the reduction and/or inhibition of the aggregation of amyloidogenic proteins, preferably of polyglutamine proteins or polyglutamine peptides.

According to the present invention this object is furthermore solved by providing tetranortriterpenoid compounds for use in the inhibition of heat shock proteins, in particular HSP40, HSP70 and HSP90.

According to the present invention this object is furthermore solved by providing tetranortriterpenoid compounds for use in increasing proteasome activity.

According to the present invention this object is solved by providing a pharmaceutical composition, comprising one or more tetranortriterpenoids, in particular selected from the group of
havanensin triacetate (S0),
khayanthone (S1),
angolensic acid methylester (S2)
3-alphahydroxy-3-deoxy angolensic acid methylester (S3),
isogedunin (S4),
epoxy (1,2 alpha) 7-deacetocy-7-oxo-deoxydihydorgedunin (S5),
1,3-dideacetyl khivorin (S6),
deacetoxy-7-oxisogedunin (S7),
1,7 -dideacetoxy-1,7-dioxokhivorin (S8),
3-beta-acetoxydeocyangoensic acid methylester (S9),
1,3-dideacetyl-7-deacetoxy-7-oxokhivorin (S10),

and salts or derivatives thereof.

According to the present invention this object is solved by providing the pharmaceutical compositions for use in the treatment, diagnosis and/or prevention of diseases as defined herein.

According to the present invention this object is solved by providing a nucleic acid, comprising the nucleotide sequence of two huntingtin fragments, wherein at least one, preferably two huntingtin fragments, is selected from huntingtin exon 1 (HDex1, wildtype) or huntingtin N-terminal fragment of amino acids 1-514 (HD514, wildtype), more preferably huntingtin exon 1 with a polyQ sequence of 17 repeats (HDex1Q17, wildtype), huntingtin exon 1 with a polyQ sequence of 68 repeats (HDex1Q68), huntingtin N-terminal fragment of amino acids 1-514 with a polyQ sequence of 17 repeats (HD514Q17, wildtype), or huntingtin N-terminal fragment of amino acids 1-514 with a polyQ sequence of 68 repeats (HD514Q68).

According to the present invention this object is furthermore solved by providing a cell, comprising a nucleic acid of the invention.

According to the present invention this object is furthermore solved by providing an in vitro method for assessing the aggregation of huntingtin in mammalian cells. The method of the invention comprises the following steps:
a) providing one or more nucleic acids, which comprise the nucleotide sequences coding for two huntingtin fragments,
b) transfecting the nucleic acid(s) into mammalian cells,
c) co-expressing the two huntingtin fragments in the transfected mammalian cells,
d) detecting the aggregation of the two huntingtin fragments.

According to the present invention this object is furthermore solved by providing a method for the identification of a compound, which modulates the aggregation of huntingtin. This method comprises the steps of the above method and further contacting the compound with the transfected mammalian cell which co-expresses the two huntingtin fragments.

According to the present invention this object is furthermore solved by providing a kit for assessing the aggregation of huntingtin, comprising a nucleic acid of the invention and optionally a cell of the invention.

### Description of the Preferred Embodiments of the Invention

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

Tetranortriterpenoid Compounds

The compounds of the present invention are natural compounds which belong to the category of tetranortriterpenoids and are characterized by a basic structure of a C₂₆ skeleton, also defined as meliacanone or angolensic acid, and a furanolactone core structure. The compounds of the present invention are derivates of meliacanone/angolensic acid. The compounds of the present invention can be isolated from species of the Meliaceae family.

The compounds of the present invention were identified during a screen of a library of natural compounds (Natural Product Collection, MicroSource Discovery Systems), wherein 11 compounds related to the group of tetranortriterpenoids were identified that affected the aggregation of polyQ expanded huntingtin in stable Tet-inducible cell lines.

The 11 identified substances (S0 to S10) showing highest effects in the aggregation process share a high structural homology (>90%) based on a similarity search using CHED (ChemDB.com).

More details are given herein below, see also Figures and Examples.

The tetranortriterpenoid is preferably selected from the group of
gedunin derivatives,
khivorin derivatives,
derivatives of angolensic acid methyl ester,
angolensic acid,
havanensin triacetate and
khayanthone.

More preferably, the tetranortriterpenoid is selected from the group of
havanensin triacetate (S0),
khayanthone (S1),
angolensic acid methylester (S2)
3-alphahydroxy-3-deoxy angolensic acid methylester (S3),
isogedunin (S4),
epoxy (1,2 alpha) 7-deacetocy-7-oxo-deoxydihydorgedunin (S5),
1,3-dideacetyl khivorin (S6),
deacetoxy-7-oxisogedunin (S7),
1,7 -dideacetoxy-1,7-dioxokhivorin (S8),
3-beta-acetoxydeocyangoensic acid methylester (S9),
1,3-dideacetyl-7-deacetoxy-7-oxokhivorin (S10),

and salts or derivatives thereof.

In a preferred embodiment of the invention, the tetranortriterpenoid is selected from havanensin triacetate (S0), khayanthone (S1), 3-alphahydroxy-3-deoxy angolensic acid methylester (S3) and isogedunin (S4),

In a further preferred embodiment of the invention, the tetranortriterpenoid is khayanthone (S1).

TABLE 1
Chemical structures and EC₅₀ values of the identified most
potent huntingtin aggregation modulators.

| | | | EC50 |
|---|---|---|---|
| | | Chemical structure | value [μM] |
| S0 | havanensin triacetate | 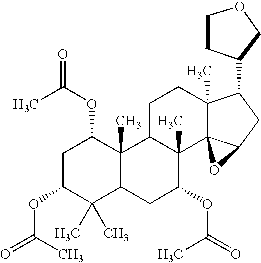 | 3 |
| S1 | khayanthone | 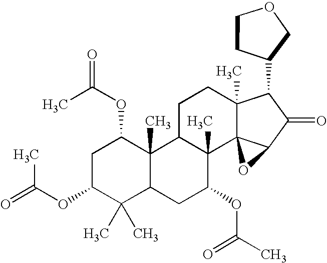 | 2 |
| S2 | angolensic acid | | 3 |
| | methylester | | |
| S3 | 3-alphahydroxy-3- deoxy angolensic acid methylester | 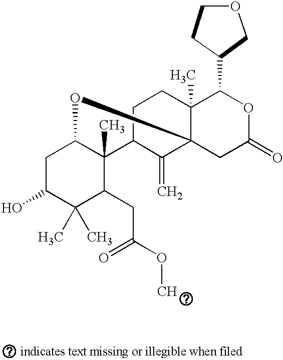 | 3 |
| S4 | isogedunin | 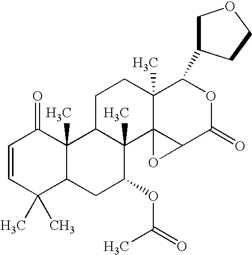 | 2 |
| S5 | epoxy (1,2 alpha) 7- deacetocy-7-oxo- deoxydihydorgedunin | 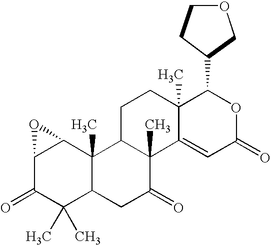 | 15 |
| S6 | 1,3-dideacetyl khivorin | 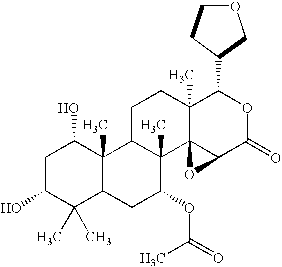 | 10 |
| S7 | deacetoxy-7- oxisogedunin | 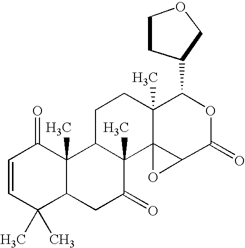 | 5 |
| S8 | 1,7-dideacetoxy- 1,7-dioxokhivorin | 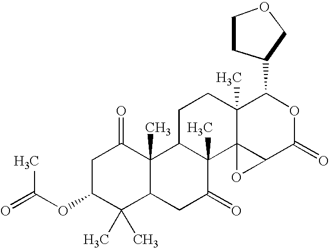 | 5 |
| S9 | 3-beta- acetoxydeocyangoensic acid methylester | 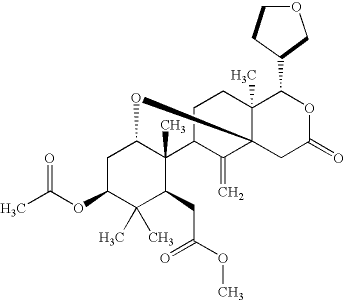 | 6 |
| S10 | 1,3-dideacetyl-7- deacetoxy-7- oxokhivorin | 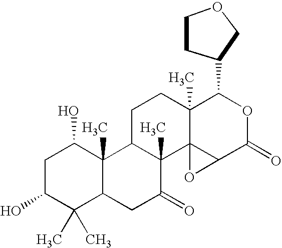 | 6 |

The medical and further uses of these compounds are described in the following.

Medical and Further Uses of the Tetranortriterpenoid Compounds

As outlined above, the present invention provides tetranortriterpenoid compounds for use in the treatment, diagnosis and/or prevention of diseases.

Preferably, the tetranortriterpenoid compounds are provided for use in the treatment, diagnosis and/or prevention of a trinucleotide repeat disorder, an amyloid disease, a neurodegenerative disease, a protein misfolding disease or a tumor

“Trinucleotide repeat disorders” (also known as trinucleotide repeat expansion disorders, triplet repeat expansion disorders or codon reiteration disorders) are a set of genetic disorders caused by trinucleotide repeats in certain genes exceeding the normal, stable, threshold, which differs per gene. The mutation is a subset of unstable microsatellite repeats that occur throughout all genomic sequences. If the repeat is present in a healthy gene, a dynamic mutation may increase the repeat count and result in a defective gene.

One group or category of trinucleotide repeat disorders are caused by a CAG repeat expansion in a protein-coding portion of specific genes. In this group/category the repeated codon is CAG, which codes for glutamine (Q). These diseases are commonly referred to as “polyglutamine (or PolyQ) diseases”. During protein synthesis, the expanded CAG repeats are translated into a series of uninterrupted glutamine residues forming what is known as a polyglutamine tract. These disorders are characterized by autosomal dominant mode of inheritance, midlife onset, a progressive course, and a correlation of the number of CAG repeats with the severity of disease and the age at onset. A common symptom of PolyQ diseases is characterized by a progressive degeneration of nerve cells usually affecting people later in life.

The polyglutamine disease is preferably selected from
Huntington's disease (HD),
dentatorubropallidoluysian atrophy (DRPLA),
spinobulbar muscular atrophy (SBMA) or
spinocerebellar ataxias (SCA), such as SCA 1, 2, 3, 6, 7 or 17,

In a preferred embodiment, the polyglutamine disease is Huntington's disease (HD).

An “amyloid disease” within this specification refers to a disease or disorder which is caused or related to the formation of amyloids or amyloid aggregates, respectively. “Amyloids” are insoluble fibrous protein aggregates sharing specific structural characteristics. Abnormal accumulation of amyloid in organs plays a role in various neurodegenerative diseases. There are two broad classes of amyloid-forming polypeptide sequences: glutamine-rich polypeptides are important in the amyloidogenesis of yeast and mammalian prions, as well as Huntington's disease. In general, for this class of diseases, toxicity correlates with glutamine content. This has been observed in studies of onset age for Huntington's disease (the longer the polyglutamine sequence, the sooner the symptoms appear). Other polypeptides and proteins such as amylin, α-synuclein in Parkinson's disease, the Alzheimer's beta protein and tau do not have a simple consensus sequence and are thought to operate by hydrophobic association. Among the hydrophobic residues, aromatic amino-acids are found to have the highest amyloidogenic propensity.

Examples of amyloid diseases are medullary carcinoma of the thyroid, systemic and organ-specific amyloidosis, Alzheimer's disease, Parkinson's disease, Huntington's disease, transmissible spongiform encephalopathy, Type 2 diabetes mellitus, yeast Prions.

A “neurodegenerative disease” within this specification refers to a condition in which nerve cells of the brain and spinal cord are progressively impaired in structure and function including death of neurons.

Recently, protein folding is beginning to be associated with ideas on protein misfolding and disease, since the structure of a protein and its ability to carry out its correct function are very tightly linked such that small structural defects can lead to a number of protein folding diseases (or “protein misfolding diseases”). These include genetic diseases such as cystic fibrosis and sickle cell anaemia, which are caused by single residue deletion and mutation respectively, rendering the protein incapable of its normal function. More recently a number of diseases have been linked to protein folding problems which lead to the build up of insoluble protein plaques in the brain or other organs. These diseases include prion diseases such as bovine spongiform encephalopathy (BSE) and its human equivalent Creutzfeld-Jakob disease (CJD), and also Alzheimer's disease, Parkinson's disease and type II (non-insulin dependent) diabetes.

As outlined above, the present invention provides tetranortriterpernoid compounds for use in the reduction and/or inhibition of the aggregation of amyloidogenic proteins, preferably of polyglutamine proteins or polyglutamine peptides.

An “amyloidogenic protein” within this specification refers to a protein which is prone to form amyloids, i.e. amyloid-forming polypeptide sequences, as defined above. For examples, see above.

Since these amyloidogenic proteins, in particular polyglutamine proteins, causes or participate in the development of protein misfolding diseases/neurodegenerative diseases/amyloid diseases/trinucleotide repeat disorders, as discussed herein and in the art, compounds that modulate the aggregation of these proteins are very suitable for the treatment, diagnosis and/or prevention of these diseases.

A preferred polyglutamine protein is huntingtin protein.

The huntingtin gene, also called HD (Huntington disease) gene, or the IT15 (“interesting transcript 15”) gene codes for a 348 kDa protein called “huntingtin protein” (htt). The gene comprises 67 exons. The HD gene is located on the short (p) arm of chromosome 4 at position 16.3. Huntingtin protein is ubiquitous, with highest levels of expression in testicles and the brain. The 5′ end of the HD gene has a sequence of 3 DNA bases, cytosine-adenine-guanosine (CAG), coding for the amino acid glutamine, that is repeated multiple times. This region is called a trinucleotide repeat. Normal persons have a CAG repeat count of between 11 and 35 repeats.

SEQ ID NO. 1 shows the complete protein sequence of huntingtin, referring to Accession No. P42858 (which is the reference sequence for a non-mutated status).

HD is caused by an unstable CAG repeat expansion in the first exon of the huntingtin gene (IT-15) which translates into an elongated polyglutamine (polyQ) stretch in the protein huntingtin. A pathological polyQ length of more than 37 glutamine residues is associated with the appearance of cytosolic, perinuclear and nuclear inclusions containing aminoterminal huntingtin fragments and sequestered proteins e.g. ubiquitin, components of the proteasome, heat-shock proteins and transcription factors (Imarisio et al., 2008). Recent evidence suggests that intermediates of the aggregation process like oligomers and protofibrils are likely to be the toxic species leading to neurodegeneration (Lansbury & Lashuel, 2006; Takahashi et al., 2008).

Thus, the compounds of the invention are preferably huntingtin aggregation modulators, which can be utilized in vitro as well as in vivo.

Preferably, the aggregated huntingtin protein is wildtype huntingtin or mutated huntingtin, such as mutated huntingtin exon 1 (HDex1), N-terminal fragment of amino acid residues 1-514 (HD514), or fragments thereof.

The suitability of the compounds of the invention for the uses and indications described can further be seen in the Examples and Figures, in particular in the FRET-based assay for the aggregation of huntingtin fragments in cells as well as in the Drosophila model of HD.

As outlined above, the present invention provides tetranortriterpernoid compounds for use in increasing proteasome activity.

There are several experimental evidences that huntingtin is cleaved by the ubiquitin-proteasome system (UPS) (DiFiglia 1997, Holmberg et al. 2004, Waelter 2001). Inhibition of proteasome activity of huntingtin expressing cells resulted in an increased number of aggregates compared to cells with a non altered proteasome activity. (Wyttenbach et al. 2000; Bence et al. 2001; Rajan et al. 2001). Furthermore, degradation of huntingtin aggregates was impeded by proteasome inhibitors applied in a transgenic mouse model. Compared to wild type huntingtin the mutated protein is more slowly degraded by the UPS pinpointing to a general impairment of the UPS (Bence et al. 2001; Rajan et al. 2001) upon presence of mutated huntingtin proteins. Therefore, the identification of biologically active substances that modify, especially increase clearance of proteins by the UPS represents a promising strategy for a treatment of HD.

In particular, it can be seen from the Figures and Examples, the compounds of the invention have an effect on proteasome activity.

As outlined above, the present invention provides tetranortriterpernoid compounds for use in the modulation of heat shock proteins, in particular HSP40, HSP70, and HSP90, respectively. A further possible therapeutic strategy in HD is the development of drugs that prevent amyloidogenesis at a very early state or induce an enhanced clearance upon the action of chaperones (Martin-Aparicio et al. 2001, Ehrnhoefer et al. 2008). In different cellular models of HD it has been shown that modulation of heat shock proteins influences the assembly of amyloid huntingtin protein (Muchowski et al. 2000; Sittler et al. 2001; Novoselova et al. 2005, Warrick et al. 2005, Cummings et al. 2001). Thus, molecules modulating heat shock proteins show promise for therapeutic intervention in HD. Furthermore, HSP40, HSP70 and HSP90, have impacts in neurodegenerative disease and cancer, thus, the tetranortriterpernoid compounds of the invention are suitable in treating said diseases etc.

In particular, it can be seen from the Figures and Examples, the compounds of the invention have an effect on the protein concentration of heat shock proteins, in particular HSP40, HSP70, and HSP90.

The tetranortriterpernoid compounds of the invention can be used/applied in the above described indications also in vitro. For example in in vitro assays, in diagnostic methods etc. The skilled artisan will be able to utilize the tetranortriterpernoid compounds of the invention in respective in vitro applications after studying the present invention.

Pharmaceutical Compositions

As outlined above, the present invention provides a pharmaceutical composition which comprises one or more tetranortriterpenoids and optionally pharmaceutically acceptable excipients and/or carriers.

The tetranortriterpenoids in the pharmaceutical compositions of the invention are preferably selected from the group of
havanensin triacetate (S0),
khayanthone (S1),
angolensic acid methylester (S2)
3-alphahydroxy-3-deoxy angolensic acid methylester (S3),
isogedunin (S4),
epoxy (1,2 alpha) 7-deacetocy-7-oxo-deoxydihydorgedunin (S5),
1,3-dideacetyl khivorin (S6),
deacetoxy-7-oxisogedunin (S7),
1,7 -dideacetoxy-1,7-dioxokhivorin (S8),
3-beta-acetoxydeocyangoensic acid methylester (S9),
1,3-dideacetyl-7-deacetoxy-7-oxokhivorin (S 10),

and pharmaceutically acceptable salts or derivatives thereof.

As outlined above, the present invention further provides the pharmaceutical compositions for use in the treatment, diagnosis and/or prevention of diseases as defined herein.

Means for Identifying Huntingtin Aggregation Modulating Compounds

As outlined above, the present invention provides nucleic acids, comprising the nucleotide sequence of two huntingtin fragments.

A “nucleic acid” refers to DNA, RNA, and derivatives thereof.

The nucleic acids of the invention comprise expression constructs, vectors, plasmids which allow the expression of the two huntingtin fragments in cells, preferably in mammalian cells.

Preferably, at least one of the two huntingtin fragments is selected from
huntingtin exon 1 (HDex1, wildtype) or
huntingtin N-terminal fragment of amino acids 1-514 (HD514, wildtype), more preferably selected from
huntingtin exon 1 with a polyglutamine sequence of 17 repeats (HDex1Q17, wildtype),
huntingtin exon 1 with a polyglutamine sequence of 68 repeats (HDex1Q68),
huntingtin N-terminal fragment of amino acids 1-514 with a polyglutamine sequence of 17 repeats (HD514Q17, wildtype), or
huntingtin N-terminal fragment of amino acids 1-514 with a polyglutamine sequence of 68 repeats (HD514Q68).

More preferably, the two huntingtin fragments are selected from
huntingtin exon 1 (HDex1, wildtype) or
huntingtin N-terminal fragment of amino acids 1-514 (HD514, wildtype), more preferably selected from
huntingtin exon 1 with a polyglutamine sequence of 17 repeats (HDex1Q17, wildtype),
huntingtin exon 1 with a polyglutamine sequence of 68 repeats (HDex 1 Q68),
huntingtin N-terminal fragment of amino acids 1-514 with a polyglutamine sequence of 17 repeats (HD514Q17, wildtype), or
huntingtin N-terminal fragment of amino acids 1-514 with a polyglutamine sequence of 68 repeats (HD514Q68).

The nucleic acids of the invention can also comprise huntingtin fragments with different numbers of polyglutamine repeats.

In one embodiment, the number of polyglutamine repeats is preferably in the range of 11 to 35 polyglutamine repeats, such as 25 Q repeats (preferably instead of 17).

In one embodiment, the number of polyglutamine repeats is preferably more than 36 polyglutamine repeats, more preferably in the range of 36 to 100 repeats, such as 70 Q repeats (preferably instead of 68).

The skilled artisan can adapt the number of polyglutamine repeats depending on the intended use/application of the nucleic acids of the invention.

Polyglutamine repeats in the range up to 35 (usually 11-35) comprise the wild-type status.

Polyglutamine repeats in the range of 36 to 39 repeats comprise probands with an increased risk to express Huntington's Disease (incomplete penetrance). Polyglutamine repeats in the range of 40 to 250 repeats comprise probands expressing the complete clinical pattern of Huntington's Disease (patients, manifestation of the disease).

Nucleic acids having repeat lengths of Q10 to Q30 are used in the state of the art/research as wild-type reference sequences. Especially, nucleic acids having repeat lengths at the borderline between non-mutated (Q30-Q35), increased risk and manifestation (Q36-Q44) are used for research to elucidate instabilities of gene locus, instabilities in genetic transmission or analysis of further gene dosage effects responsible for the disease onset. Nucleic acids having repeat lengths of Q40 to Q80 are used to study the disease phenotype with highest prevalence in the patient group. Furthermore, nucleic acids having largely extended repeat lengths >Q60 are used to study juvenile onsets of Huntington's Disease and further gene dosage effects. Thus, the nucleic acids of the present invention can be designed and applied accordingly.

SEQ ID NO. 1 shows the complete protein sequence of huntingtin, referring to Accession No. P42858 (reference sequence for a non-mutated status), of which an N-terminal part (aa 1-90) was used to generate the huntingtin exon 1 (HDex1) constructs:

The amino acid sequence of HDex1Q17 constructs have the following sequence [SEQ ID NO. 2]:

matleklmka feslksf (q)₁₇ pppppppppp pqlpqpppqa
qpllpqpqpp ppppppppgp avaeeplhrp

Note that CAG repeat number coding for glutamine varies between the deposited sequence (P4285 8; which has 23 repeats of CAG) and the cloned HDex1Q17 construct (which has 17 repeats of CAG).

The amino acid sequence of SEQ ID NO. 3 is present in HDex1Q68 constructs according to protein sequence (AccNr. P42858):

matleklmka feslksf (q)₆₈ pppppppppp pqlpqpppqa
qpllpqpqpp ppppppppgp avaeeplhrp

Thus, the nucleotide sequence of HDex1Q17 codes for the amino acid sequence of SEQ ID NO. 2 and the nucleotide sequence of HDex1Q68 codes for the amino acid sequence of SEQ ID NO. 3.

The nucleotide sequence of HD514Q17 as well as HD514Q68 can be derived from the nucleotide sequence that codes for amino acids 1-514 of the amino acid sequence of SEQ ID NO. 1. Again, the CAG repeat number coding for glutamine varies between the deposited sequence (P42858; SEQ ID NO. 1 which has 23 repeats of CAG) and the cloned HD514Q17 construct (which has 17 repeats of CAG) as well as the cloned HD514Q68 construct (which has 68 repeats of CAG). The last amino acid of HD514Q17 as well as HD514Q68 is amino acid 514 of SEQ ID NO. 1.

The orientation and localization of the two huntingtin fragments on the nucleic acids of the invention can be differed, but will allow that both huntingtin fragments are expressed in a cell, preferably a mammalian cell. Depending on the choice of promoter and other regulating elements, which are known to the skilled artisan, the huntingtin fragments
are adjacent to each other or have linker and other sequences (e.g. promoter) in-between or overlap;
are oriented in the same direction and in different directions;
are under the control of the same promoter or different promoters.

In a preferred embodiment, the nucleic acid has the form of a bidirectional construct, which is preferably an expression construct, vector. Bidirectional means that the coding sequences of the huntingtin fragments are oriented in different, i.e. opposite directions.

See FIG. 1B.

The nucleic acid, in particular the bidirectional construct, furthermore preferably comprises a Tet-regulated promoter, wherein the Tet-regulated promoter preferably comprises a tetracyclin responsive element (TRE) and CMV promoter(s).

Preferably, the nucleic acids of the invention allow the simultaneous expression of the two huntingtin fragments under the control of a single tetracycline response element.

In this embodiment, the nucleic acids contain a bidirectional promoter—a TRE containing the tet operator sequences flanked by two identical minimal cytomegalovirus promoters in opposite orientations. When this bidirectional construct is stably integrated into a cell line expressing the tetracycline-controlled transactivator (tTA) or reverse tTA (rtTA), expression of both cloned genes (i.e. the two huntingtin fragments) is co-regulated by tetracycline or its derivative, doxycycline.

In a preferred embodiment, each of the two huntingtin fragments is a fusion protein with a chromophor, in particular with a fluorophor. Wherein “fusion protein” means that both parts of the fusion, i.e. the huntingtin and the chromophor, are preferably expressed such that they are linked to each other.

The fluorophor is preferably green fluorescent protein (GFP) or a derivate of GFP or is enhanced green fluorescent protein (EGFP) or a derivate thereof.

In particular, the fluorophor is cyan fluorescent protein (CFP) or yellow fluorescent protein (YFP), more preferably enhanced cyan fluorescent protein (ECFP) or enhanced yellow fluorescent protein (EYFP).

Further derivatives of GFP/EGFP and further fluorophors are known in the art, such as blue fluorescent protein (EBFP), red fluorescent protein (DsRed) and its derivatives.

Preferably, each of the two huntingtin fragments is fused to a different fluorophor, preferably to fluorophors that are a FRET (fluorescence resonance energy transfer) pair, preferably CFP and YFP (ECFP and EYFP). FRET and fluorophors that form suitable FRET-pairs are known in the art.

Preferably, the chromophor is N-terminal or C-terminal fused to each of the two huntingtin fragments, in particular C-terminal.

In a preferred embodiment the nucleic acid of the invention comprises
HDex1Q17-YFP and HDex1Q17-CFP,
HDex1Q68-YFP and HDex1Q68-CFP,
HD514Q17-YFP and HD514Q17-CFP, or
HD514Q68-YFP and HD514Q68-CFP,
or comprising at least HDex1Q68-YFP, preferably in combination with HDex1Q17-CFP, HD514Q17-CFP or HD514Q68-CFP.
or comprising at least HDex1Q68-CFP, preferably in combination with HDex1Q17-YFP, HD514Q17-YFP or HD514Q68-YFP.

Thus, preferred combinations of two hunting fragments fused to CFP or YFP are:

| YFP fusion | CFP fusion |
|---|---|
| HDex1Q17-YFP | HDex1Q17-CFP |
| HDex1Q68-YFP | HDex1Q68-CFP |
| HD514Q17-YFP | HD514Q17-CFP |
| HD514Q68-YFP | HD514Q68-CFP |
| HDex1Q68-YFP | HDex1Q17-CFP |
| HDex1Q68-YFP | HD514Q17-CFP |
| HDex1Q68-YFP | HD514Q68-CFP |
| HDex1Q17-YFP | HDex1Q68-CFP |
| HD514Q17-YFP | HDex1Q68-CFP |
| HD514Q68-YFP | HDex1Q68-CFP |

More preferably, the nucleic acid comprises at least HDex1Q68, such as HDex1Q68-YFP or HDex1Q68-CFP. HDex1Q68 is preferred due to its fast and efficient aggregation characteristics, which is very suitable for the methods of the invention described below.

As outlined above, the present invention provides a cell which comprises at least one nucleic acid of the invention.

Preferably, the cell expresses the two huntingtin fragments. Preferably, the expression is stably inducible.

Preferred cells are mammalian cells, such as CHO and others, which are known in the art.

In a preferred embodiment the cell is a cell of a Tet-off cell line. These cell lines are suitable for utilizing the bidirectional constructs with Tet-regulated promoter, TRE element as described above and in the Examples. Such cell lines are commercially available.

As outlined above, the present invention provides an in vitro method for assessing the aggregation of huntingtin in mammalian cells.

The method of the invention preferably comprises the following steps:
a) providing one or more nucleic acids, which comprise the nucleotide sequences coding for two huntingtin fragments,
b) transfecting the nucleic acid(s) into mammalian cells,
c) co-expressing the two huntingtin fragments in the transfected mammalian cells,
d) detecting the aggregation of the two huntingtin fragments.

The nucleic acid(s) of step a) are either two nucleic acids, wherein each comprises the nucleotide sequence coding for one of the two huntingtin fragments, or is one nucleic acid, which comprises both huntingtin fragments (preferably a nucleic acid of the present invention, as described above).

Preferably, the huntingtin fragments are selected from huntingtin exon 1 (amino acids 1-90) (HDex1, nucleotide sequence encoding SEQ ID NO. 2) and huntingtin N-terminal fragment of amino acids 1-514 (HD514, nucleotide sequence encoding amino acids 1-514 of SEQ ID NO. 1), as described above.

Further, the huntingtin fragments comprise a polyglutamine sequence (polyQ sequence). In one embodiment, the number of polyglutamine repeats is preferably in the range of 11 to 35 polyglutamine repeats, in particular a polyQ sequence of 17 repeats (Q17) or 25 repeats.

In one embodiment, the number of polyglutamine repeats is preferably more than 36 polyglutamine repeats, more preferably in the range of 36 to 100 repeats, in particular a polyQ sequence of 68 repeats (Q68) or 70 repeats.

Thus, the huntingtin fragments can have different numbers of polyglutamine repeats, as described in detail above. The skilled artisan can adapt the number of polyglutamine repeats, for example depending on the desired rate of aggregation and other factors.

However, one of the two huntingtin fragments is preferably huntingtin exon 1 with a polyQ sequence of 68 repeats (HDex1Q68, nucleotide sequence encoding SEQ ID NO. 3), since HDex1Q68 has fast and efficient aggregation characteristics.

The huntingtin fragments can comprise
the same polyQ sequence,
   such as both fragments comprise a Q17 sequence or
   both fragments comprise a Q68 sequence,
different polyQ sequences,
   such as a Q17 sequence and a Q68 sequence.

Preferably, each of the two huntingtin fragments is a fusion protein with a chromophor, in particular with a fluorophor, as described above.

The preferred fluorophors and the possible fusion proteins are also described herein above.

C-terminal fusions of GFP derivatives as chromophor/fluorophor to the huntingtin fragment are preferred due to preferred aggregation characteristics of the fusion proteins.

In a preferred embodiment, the nucleic acid in step a) is a nucleic acid of the invention, i.e a nucleic acid comprising the nucleotide sequences of two huntingtin fragments, as described above, wherein the nucleic acid preferably comprises at least HDex1Q68, such as HDex1Q68-YFP or HDex1Q68-CFP.

In a preferred embodiment, the detection of the aggregation in step d) is carried out by measuring the fluorescence resonance energy transfer (FRET) signal.

In this embodiment the two huntingtin fragments are fused to fluorophors that form a FRET pair and which is suitable for measuring their fluorescence emission and thus, the FRET, in cells, preferably via live cell imaging.

Preferably suitable is a nucleic acid of the invention, i.e a nucleic acid comprising the nucleotide sequences of two huntingtin fragments fused to (preferably) CFP/YFP, as described above.

FRET will occur when the two fluorophors are in close proximity, i.e. when the two huntingtin fragments form aggregates.

The rate and other characteristics of the aggregation process can be detected and measured via the monitoring/measuring of the FRET signal, as it is known to the skilled artisan.

Preferably, a cell according to the present invention is used as a mammalian cell in step b).

The cells are preferably stably inducible cell lines.

As outlined above, the present invention provides a method for the identification of a compound, which modulates the aggregation of huntingtin.

This method comprises the steps of the above method and further comprises the step of contacting the compound with the transfected mammalian cell which co-expresses the two huntingtin fragments. Preferably, the compounds are added to the respective cell culture.

In the preferred embodiment, wherein the aggregation of huntingtin in step d) is detected via FRET, as described above, the nucleic acids of the invention are very suitable, wherein such a nucleic acid preferably comprises at least HDex1Q68, such as HDex1Q68-YFP or HDex1Q68-CFP.

Preferably, the method comprises the step of determining the autofluorescence of the compound to be tested.

A compound that modulates the huntingtin aggregation by reducing or inhibiting it, the FRET signal will decrease or disappear after the compound has been contacted with the respective cell.

A compound that modulates the huntingtin aggregation by increasing it, for instance by increasing the rate of aggregation, the FRET signal can be detected at an earlier timepoint. The skilled artisan will be able to apply this method after studying this specification.

Preferably, the method further comprises the step of determining the cell viability. Preferably, the cell viability is tested fluorometrically. Such cell viability tests are known in the art. The cell viability testing will reveal if tested compounds are cytotoxic compounds, which can then be excluded from further studies and evaluations.

For more details, see also the Examples.

Selection criteria for potential active compounds can be predetermined or preset, for example a percentage of the reduction of the FRET value and/or a percentage of maximal reduction of the cell number.

In a preferred embodiment the method comprises the following steps:
a) providing one or more nucleic acids, which comprise the nucleotide sequences coding for two huntingtin fragments, wherein each of the huntingtin fragments is preferably fused to a fluorophor,
b) transfecting the nucleic acid(s) into mammalian cells, co-expressing the two huntingtin fragments in the transfected mammalian cells,
c) contacting the compound with the transfected mammalian cell which co-expresses the two huntingtin fragments,
e) detecting the aggregation of the two huntingtin fragments, preferably by measuring the FRET signal,
f) determining the cell viability, preferably fluorometrically.

This method was used by the inventors to screen a natural compound library and led to the identification of the tetranortriterpernoid compounds of the invention, which modulate huntingtin aggregation by reducing/inhibiting it.

For more details, see also the Examples.

As outlined above, the present invention provides a kit for assessing the aggregation of huntingtin. The kit comprises the nucleic acid(s) of the invention and optionally the cell(s) of the invention.

In the present invention, the inventors established a cellular FRET-based model for the aggregation of mutated huntingtin using stable Tet-inducible cell lines expressing both CFP- and YFP-labelled huntingtin exon 1 fragments. The model was used to screen a library of natural compounds (Natural Product Collection, MicroSource Discovery Systems). The inventors identified 11 compounds related to the group of tetranortriterpenoids that affected the aggregation of polyQ expanded huntingtin in the stable Tet-inducible cell lines. The most effective compound, khayanthone, improves significantly motor deficits in a transgenic Drosophila model for Huntington's Disease (HD).

The following drawings and examples illustrate the present invention without, however, limiting the same thereto.

### BRIEF DESCRIPTION OF THE DRAWINGS

FIG. 1. Principle and procedure of the cellular aggregation assay.

(A) Fluorescence microscopy images of stable inducible CHO—tet off cell lines expressing simultaneously wildtype (CHO-AA8/Q17-Q17) or mutant (CHO-AA8/Q68-Q68) huntingtin fused to CFP or YFP. Read out of the assay is based on the measurement of FRET between CFP and YFP in mutant huntingtin aggregates. FRET is expressed as FRET efficiency E % (colour coded bar).

(B) Schematic representation of the inducible bidirectional expression construct. Mutant huntingtin proteins fused either to ECFP or to EYFP. The expression of both proteins is induced simultaneously by the removal of doxicycline from the culture medium, which otherwise binds to the tetracycline response element (TRE) and represses the protein expression.

(C) Flow chart summarizing the assay procedure. FRET values represent the rate of mutant huntingtin aggregates. Toxic compounds are excluded by propidium iodide (PI) testing.

FIG. 2. Assay validation and compound library screen.

(A/B) Mutant huntingtin expressing cell line (CHO-AA8/Q68-Q68) treated with different benzothiazoles. PGL-135 and PGL-137 reduced mutant huntingtin aggregates which is indicated by a decrease of the rel. FRET value (F_{A}), whereas the cell viability was not affected. Error bars represent the SD of 3 data points. Arbitrary units (a.u.)

(C) Immunodetection of mutant huntingtin aggregates upon PGL-135 treatment of cells using a filter retardation assay. Increasing concentrations of PGL-135 result in a significant decrease of mutant huntingtin aggregates.

FIG. 3. Effects of khayanthone in the FRET assay.

(A) Fluorescence microscopy images of CHO-AA8/Q68-Q68 cells treated with khayanthone. Number of mutant huntingtin aggregates decrease with increasing compound concentration. Scale bar corresponds to 100 μm.

(B) Dose response curve of stable CHO-AA8/Q68-Q68 cells treated with khayanthone (3-50 μM). The rel. FRET value (-▪-) decreases whereas the cell number (-□-) is unaffected. Error bars represent the SD of 6 data points. Arbitrary units (a.u.).

FIG. 4. Concentration-dependent effect of khayanthone on proteasome.

The concentration-dependent effect of khayanthone, 17-AAG, Gedunin, and Lactacystin on proteasome activity was tested using a non-recombinant CHO Tet-Off cell line. Cells were incubated with the given compounds for 24 h. Subsequently, proteasome activities were measured by a fluoroscan according to the degradation rate of a fluorogenic substrate (Suc-LLVY-AMC). In correlation to DMSO treated controls khayanthone led to an increase of proteasome activity about 40-50% in CHO-AA8/Tet-Off cells.

FIG. 5. Effect of khayanthone on the protein concentration of HSP40, HSP70 and HSP90.

Utilizing Western-blot analysis concentration-dependent effects of khayanthone on protein expression of HSP40, HSP70, and HSP90 were detected. Immuno detection was performed using HSP specific monoclonal antibodies, respectively. Increase of khayanthone administration leads to a decreased expression of tested heat shock proteins.

FIG. 6. Khayanthone improves motor activities in a Drosophila model of HD.

(A) Expression of the htt proteins in transgenic flies is driven by the bipartite expression system upstream activator sequence (UAS)-GAL4 (yeast transcriptional activator). Stocks w; P(w^{+mC}; w+; elav−GAL4/CyO) and w; P(w+mC=UAS-Q93httexon1) were crossed in order to obtain flies expressing mutant htt fragments in all neurons.

(B, C) Flies expressing Htt-Exon-1Q93 were fed with khayanthone (100 μM, 250 μM) or DMSO supplemented food. Effects of khayanthone on flies' motor ability were measured with the climbing assay. For this purpose flies were placed in a vial, then the height half of them were able to climb from the bottom within 60 sec was determined. In addition the time was measured which half of them needed to climb up 6 cm. With increasing age flies treated with khayanthone maintain an improved mobility compared to DMSO treated flies. Each curve represents a population of ˜40 flies. Error bars represent the SD of 5 repeats on each measuring point.

### EXAMPLES

Methods

Compounds

Compounds tested were solved in DMSO as 100 mM (Indomethacin, Coenzyme Q10), 50 mM (Congo red), 25 mM (NDGA), 10 mM (Scriptaid, PGL-135, Riluzole), 1 mM (Chrysamin G, Half Chrysamin G, Creatine, z-VAD-FMK, Mithramycin, Diclofenac sodium), 0.05 mM (Geldanamycin) solution or in water as 4000 mM (Sodium salicylate), 100 mM (Threhalose, Creatine), 10 mM (Cystamine Dihydrochloride, Y-27635). All substances except Coenzyme Q10 and Creatine (Sigma Aldrich) were purchased from Calbiochem.

Plasmid Constructs

Huntingtin fusion proteins (Q17-YFP, Q68-YFP, Q17-CFP, Q68-CFP) coding for sequences of N-terminal huntingtin (aa1-90) with 17 and 68 polyglutamines, respectively, were PCR amplified using HD514Q17 and HD514Q68 constructs (Sigler et al., 2003) and cloned into the pBI cloning system (Clontech) including a bidirectional tet-responsive promoter.

SEQ ID NO. 1 shows the complete protein sequence of huntingtin, referring to Accession No. P42858 (reference sequence for a non-mutated status), of which an N-terminal part (aa 1-90) was used to generate the HDex1 constructs:

The amino acid sequence of HDex1Q17 constructs have the following sequence [SEQ ID NO. 2]:

matleklmka feslksf (q)₁₇ pppppppppp pqlpqpppqa
qpllpqpqpp ppppppppgp avaeeplhrp

Note that CAG repeat number coding for glutamine varies between the deposited sequence (P42858; 23×CAG) and the cloned HDex1Q17 construct (17×CAG).

The amino acid sequence of SEQ ID NO. 3 is present in HDex1Q68 constructs according to protein sequence (AccNr. P42858)

matleklmka feslksf (q)₆₈ pppppppppp pqlpqpppqa
qpllpqpqpp ppppppppgp avaeeplhrp

Primers used for amplication of huntingtin ex1 and C-terminal fusion of GFP variants:

[SEQ ID NO. 4]

Htt-EcoRI-f: CGCGAATTCCATGGCGACCCTGGAAAAGC

[SEQ ID NO. 5]

Httex1-BamHI-r: CGCGGATCCTTTGGTCGGTGCAGCGGCTCCT

Primers used for amplication of huntingtin ex1 and N-terminal fusion of GFP variants:

[SEQ ID NO. 6]

Htt-EcoRI-f: CGCGAATTCCATGGCGACCCTGGAAAAGC

[SEQ ID NO. 7]

Httex1-BamHI-Stop-r: CGCGGATCCTCATGGTCGGTGCAGCGG
CTCCT

Generation of Stable, Inducible Cell Lines

Tet off CHO-AA8 cells (Clontech) stably expressing tTA, were cotransfected with 0.75 μg linearized huntingtin construct and 0.75 μg linearized TRE2-hyg (Clontech) using Fugene 6 (Roche). The cells were selected in Ham's F12 medium (PAA) with 10% fetal calf serum (PAA), 200 μg/mL hygromycin B (Invitrogen), 1.6 ng/mL doxicycline (Clontech). After two weeks selection, the cells where diluted into 10 cm dishes, and single clones isolated according to the “scratch and sniff” protocol (Karin, 1999). Transgene expression of inducible and non-inducible cells was verified by Western blot analysis and fluorescence microscopy. The cells were maintained in Ham's F12 Media with 8 ng/mL doxicycline and 0.1 mg/mL hygromycin over 48 hours to supress expression of htt fusion proteins.

Western Blot Analysis and Filter Retardation Assay

CHO-k1 cells were transiently transfected with 1 μg plasmid DNA with FuGene6 (Roche) and lysed after 48 hours with ice cold NP-40 lysis buffer (50 mM Tris/HCl, 150 mM NaCl, 50 mM NaF, 0.5% NP40, 1 mM PMSF, protease inhibitor cocktail (Sigma)). In case of testing the inducibility of the stable huntingtin expressing tet off CHO AA8 cells, the cells treated with 1.6 ng/mL doxicycline, without doxicycline and without doxicycline/2% DMSO and lysed after different time points.

The filter retardation assay was accomplished as described (Scherzinger et al., 1999). Cells were lysed (100 mM NaCl, 5 mM MgCl2, 0.5% NP-40, 1 mM EDTA, 50 mM Tris-HCl, protease inhibitors, 25 U/mL benzonase, pH 8.8), denatured 5 min at 95° C. with 2% SDS/50 mM DTT and cell lysates equal 2 μg protein were filtered through a 0.2 μm cellulose acetate membrane (Schleicher & Schuell) using a dot blot unit. The immunodetection was performed using the same antibody detection system as described above. Quantitative determination of the relative aggregate amount was evaluated with the Lumi Imager F1 System and Lumi Analyst 3.0 (Boehringer Mannheim, Germany).

FRET Microscopy

FRET efficiency was determined using the acceptor photobleaching method (Kenworthy 2001). Briefly, widefield images of cells were made using a Zeiss Axiovert 200M microscope, a 63× objective, a Zeiss Axiocam MRm camera and appropriate filter sets for CFP and YFP, respectively. Images were captured before and after bleaching of the acceptor with the YFP filter set for 5 minutes. After background subtraction FRET images were calculated: E=1−(CFP_{before bleaching}/CFP_{after bleaching}).

Cellular FRET Assay

CHO-AA8/Q68-Q68, CHO-AA8/Q17-Q17 and CHO-AA8 cells were seeded in 96 well plates. After attachment of the cells, the medium was exchanged for Ham's F 12 medium containing 10% doxicycline free FBS and 2% DMSO supplemented with compounds. After 48 h the fluorescence intensities of cells were measured using a fluorescence plate reader (Fluoroscan Ascent, Thermo Scietific) and filtersets for CFP (ex 444 nm, em 485 nm), YFP(ex 485 nm, em 538 nm) and FRET (ex 444 nm, em 538 nm). After correction of background fluorescence (subtraction of CHO-AA8 cell fluorescence intensities) the FRET signal was determined using the method: F_{A}=(FRET-a*YFP)/CFP with a=0.11 (Zal u. Gascoigne 2004, Zal et al. 2002). In following, the viability of cells was measured using a propidium iodide exclusion assay (Sattler et al. 1997). PI was added to the cells at final concentration of 0.15 mg/ml. After 15 minutes the PI fluorescence intensity was measured (ex 530 nm, em 620 nm). The cells were then lysed with 0.5% Triton X-100 (TX100) and the PI fluorescence intensity was measured again. Viability of cells was calculated as viability=1−(PI_{before TX100}/PI_{after TX100}).

Library-Screen

The Natural Product Collection from MicroSource Discovery Systems, Inc. was screened, which consists of 720 naturally derived compounds.

To determine the potential of possibly inhibiting substances the inventors performed dose response assays at concentrations ranging from 1, to 100 μM.

Screening was performed in 96-well plates (greiner bio-one) containing 1×10⁴ cells/well of the stably inducible tet off cell lines htt-mut (six-fold) and CHO-AA8 line (three fold) for background control. Simultaneously, each compound was checked for its potential autofluorescence.

During the screen, the inventors selected for compounds which showed a reduced FRET signal of 30% and a cell viability higher than 85%. Thirty seven compounds, meeting this criteria, were selected to be retested in a second screen at a concentration of 10 μM.

Seven compounds showed a reduction of the FRET signal with no toxic effects at concentrations 10, 3 and 1 μM.

Results

FRET Value Represents Polyglutamine Aggregates of Mutant Huntingtin

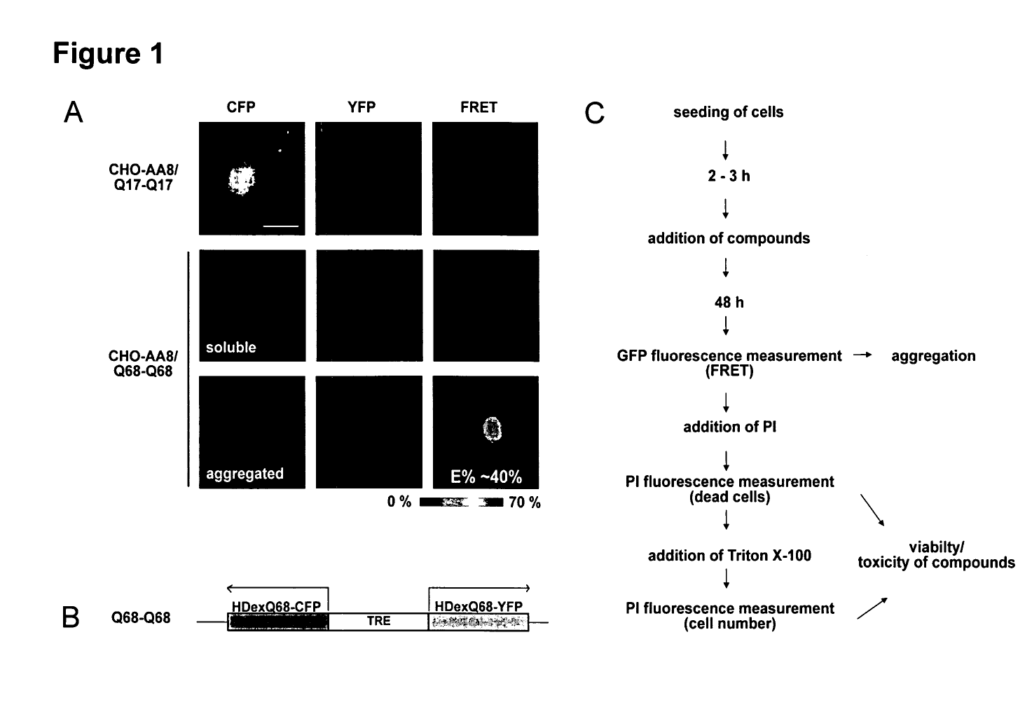

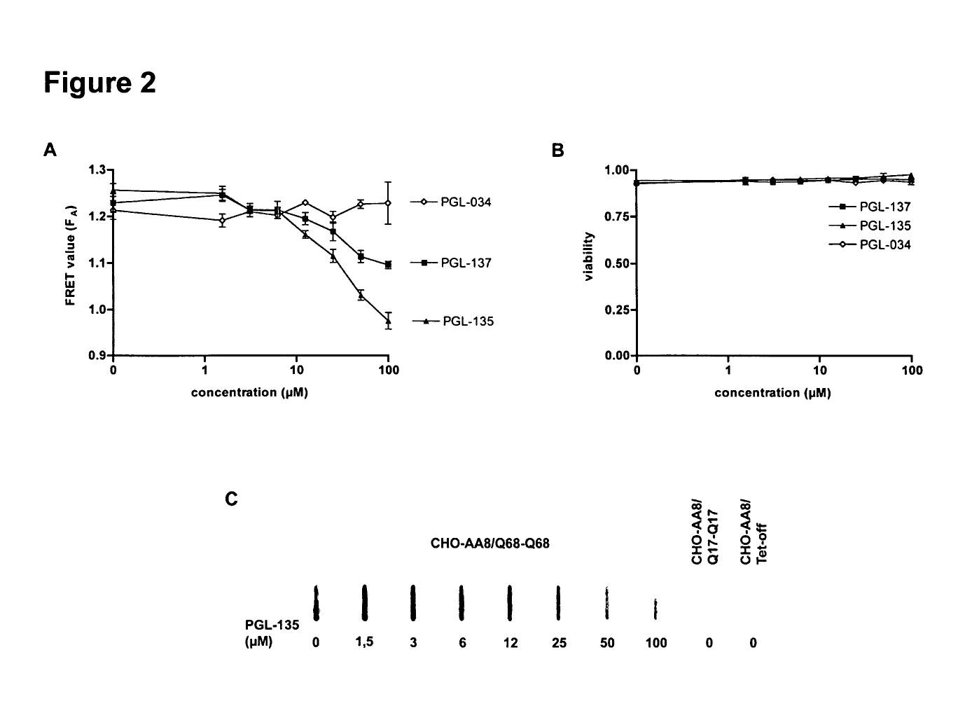

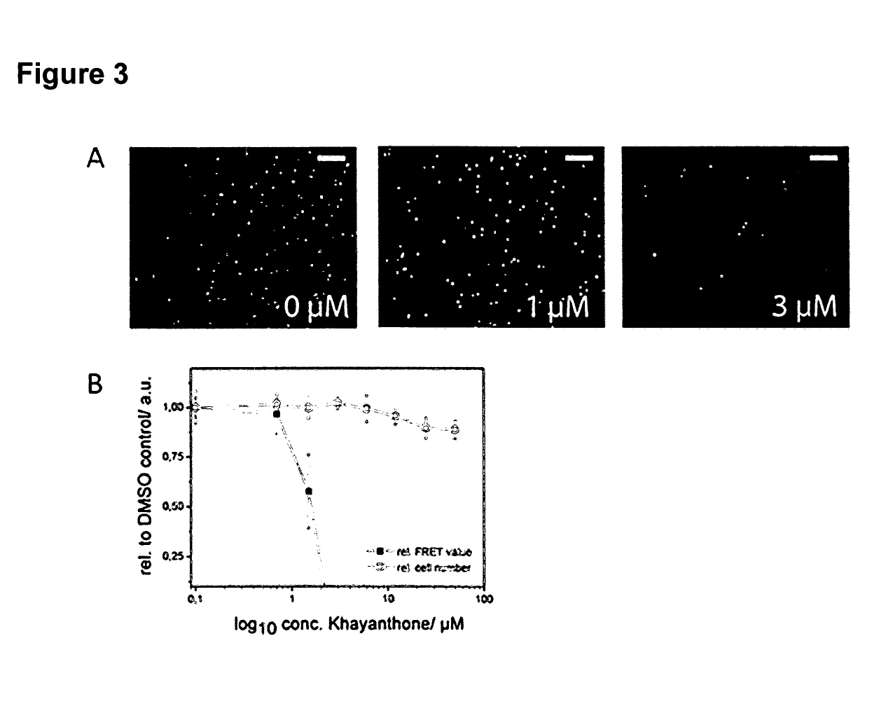

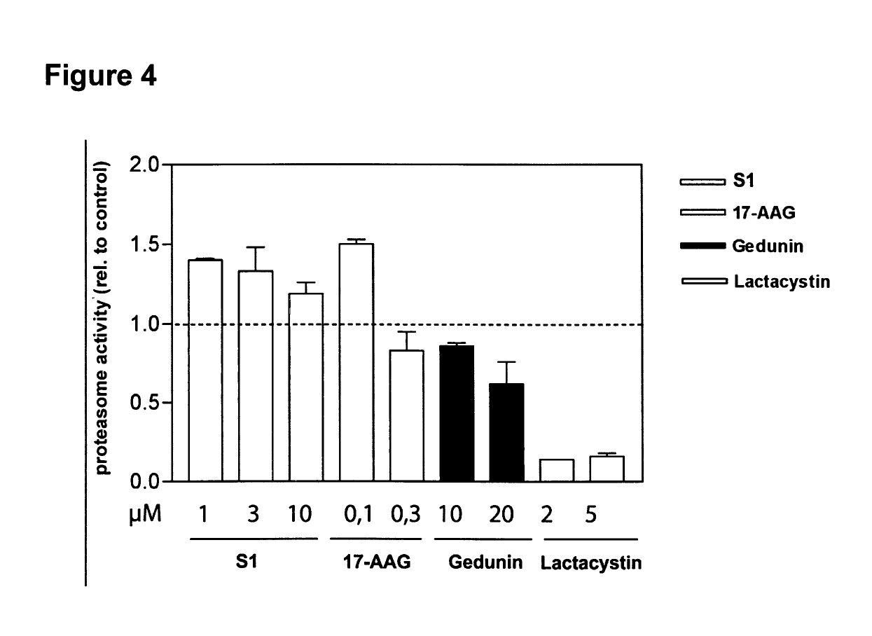

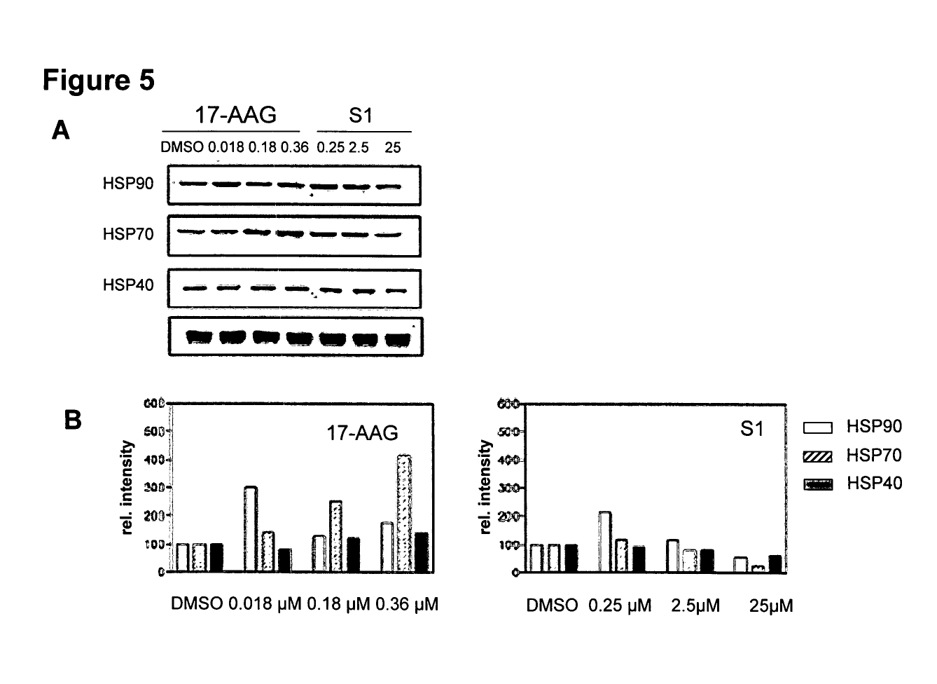

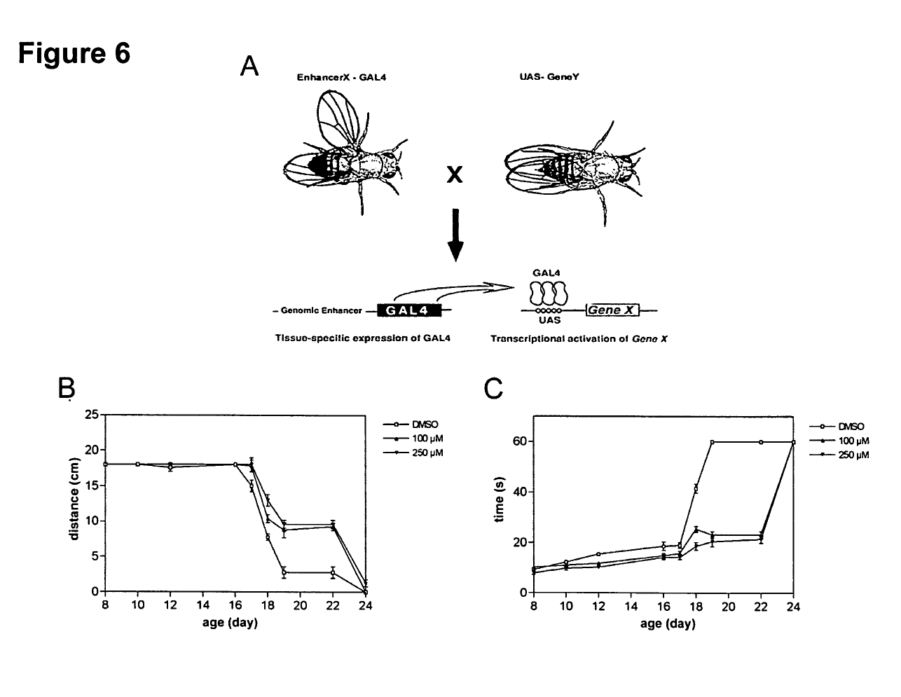

Wildtype and mutant huntingtin fragments with 17 or 68 polyglutamines were fused each with ECFP and EYFP. After co-transfection of the mutant (HDexQ68-ECFP and HDexQ68-EYFP) and wildtype huntingtin (HDexQ17-ECFP and HDexQ17-EYFP) fusion proteins in CHO-K1 cells FRET measurements on the single cell level were performed with different microscopy methods (3-cube-imaging, acceptor-bleaching, fluorescence life time measurement (FLIM)). Only aggregated mutant huntingtin fragments had a reduced life time of τ=1.25 ns, which is equivalent to a FRET efficiency of E=43%. Whereas cytosolic soluble wildtype and mutant huntingtin fragments revealed the same fluorescence live times (τ=2.2 ns) as the ECFP control (FIG. 1A) which means that no FRET efficiency was detectable (Elangovan et al., 2002; Pollitt et al., 2003).

Assay Development and Validation Using Selected Compounds

Two stable inducible CHO Tet off cell lines were constructed expressing simultaneously wildtype (CHO-AA8/Q17-Q17) or mutant (CHO-AA8/Q68-Q68) huntingtin fragments fused to ECFP or EYFP (FIG. 1B) after withdrawal of doxicycline (dox). Basal and induced expression levels of huntingtin fusion proteins were examined by Western blot analysis at different time intervals (4, 24, 48, and 72 hours). Dox showed a clear inhibition of expression, whereas removal of dox induced the expression (data not shown). For the screening approach stable inducible huntingtin expressing cell lines were seeded in 96-well plates and the fluorescence intensities were measured with a fluorescence plate reader. Apparent FRET values were calculated according to the 3-cube method (Zal 2002).

The cellular aggregation assay was validated and characterized by 20 compounds selected from literature, described as biologically active in polyQ diseases (see Table 2) and showing different modes of action (antioxidant, HSP90 inhibitor, anti-inflammatory, HDAC inhibitor, binding on amyloid inclusions).

TABLE 2
Compounds for validation of the cellular aggregation assay.

| | | | | | Effect in other in vitro or in |
|---|---|---|---|---|---|
| | | | | | vivo models |
| Substance | Remarks | FRET-signal | Viability | Cell number | (reference) |
| congo red | azo-dye, amyloidophil | ↓ ≧30 μM | no effect | ↓ ≧125 μM | (Apostol et al., 2003; Heiser et |
| | | | | | al., 2000; Sanchez et al., 2003; |
| | | | | | Smith et al., 2001) |
| chrysamine G | congo red analog | no effect | no effect | no effect | (Heiser et al., 2000; Smith et al., |
| | | | | | 2001) |
| half-Chrysamine G | congo red analog | no effect | no effect | no effect | — |
| geldanamycin | benzoquinone, antibiotic | no effect | no effect | no effect | (Sittler et al., 2001) |
| 17-AAG | geldanamycin derivativ | ↓ ≧0.3 μM | ↓ ≧0.3 μM | ↓ ≧0.3 μM | (Waza et al., 2005) |
| Riluzole | benzothiazole | no effect | no effect | no effect | (Heiser et al., 2002) |
| PGL-034 | benzothiazole | no effect | no effect | no effect | (Heiser et al., 2002) |
| PGL-135 | benzothiazole | ↓ ≧12.5 μM | no effect | no effect | (Heiser et al., 2002) |
| PGL-137 | benzothiazole | ↓ ≧12.5 μM | no effect | no effect | (Heiser et al., 2002) |
| Diclofenac | NSAID, COX-inhibitor | no effect | no effect | ↓ ≧250 nM | — |
| sodiumsalicylat | NSAID, COX-inhibitor | no effect | no effect | ↓ ≧125 μM | (Ishihara et al., 2004) |
| Indomethacin | NSAID, COX-inhibitor | ↓ ≧1.25 μM | ↓ 10 μM | ↓ ≧1.25 μM | (Ishihara et al., 2004) |
| NDGA | NSAID, LOX-inhibitor | no effect | no effect | ↓ 500 nM | (Ishihara et al., 2004) |
| Y-27632 | ROCK-I/II-inhibitor | no effect | no effect | no effect | (Pollitt et al., 2003) |
| Scriptaid | HDAC-inhibitor | ↑ ≧25 μM | ↓ ≧25 μM | ↓ ≧25 μM | (Corcoran et al., 2004) |
| Trehalose | disaccharide | no effect | no effect | no effect | (Tanaka et al., 2004; Tanaka et |
| | | | | | al., 2005) |
| Cystamine | transglutaminase- | no effect | no effect | no effect | (Igarashi et al., 1998; Karpuj et |
| | inhibitor | | | | al., 2002) |
| Creatine | organic acid, synthesised | no effect | no effect | no effect | (Andreassen et al., 2001; |
| | in kidney, liver and | | | | Dedeoglu et al., 2003; Ferrante |
| | pancreas | | | | et al., 2000) |
| coenzyme Q10 | endogeneous cellular | no effect | no effect | no effect | (Ferrante et al., 2002; Smith et |
| | antioxidant | | | | al., 2006) |
| C2-8 | sulfobenzoe acid | ↓ ≧3 μM | ↓ 50 μM | ↓ ≧6 μM | (Zhang et al., 2005) |
| | derivativ | | | | |

HDAC = Histonedeacetylase;
ROCK = Rho-associated protein kinase;
COX = Cyclooxygenase;
LOX = Lipoxygenase;
NSAID = non steroidal anti-inflammatory drug

Because most test compounds were dissolved in 100% DMSO the inventors investigated if the DMSO concentration influenced the Tet off inducible expression. Therefore, the inventors diluted several DMSO concentrations (0-5%) in the cell culture medium and measured the expression of HDexQ17-EYFP in the CHO-AA8/Q17-Q17 cell line with a fluorescence plate reader. A concentration up to 2.5% DMSO provoked a 7-fold fluorescence induction, higher DMSO concentrations were toxic (data not shown). For this reason it was important to keep the DMSO concentration constant during the assay procedure.

The test compounds were mixed in a fresh doxicycline-free medium with 2% DMSO and added to the Tet off cell lines two to three hours after seeding and attachment of the cells. This medium exchange step was further essential for the optimal induction of the transgenic expression (6-7 fold) (Rennel and Gerwins, 2002).

The FRET-measurement was performed 48 h later. The difference between the apparent FRET value F_{A} of the CHO-AA8/Q68-Q68 (1.2-1.4) and the CHO-AA8/Q17-Q17 cells (0.9-0.95) reflects the assay range (100% protein aggregates-0% aggregates). Cytotoxic compounds were identified by a propidium iodide-dead cell staining (FIG. 1C).

The azo-dye congo red is a well-known reference compound for the detection and reduction of amyloid inclusions in different in vitro and in vivo models. Congo red showed clear inhibitory effects (>30 μM) in our cellular aggregation assay (Table 2) (Apostol et al., 2003).

The benzothiazoles PGL-135 (>12.5 μM) and PGL-137 (>25 μM) also reduced the huntingtin aggregates without affecting the cell viability (FIG. 2A/B). The coherence between decreasing FRET values and less polyglutamine aggregates in PGL-135 treated cells was further validated by fluorescence microscopy imaging (data not shown) and a filter retardation assay (Heiser et al., 2000) (FIG. 2C).

The congo red analogue half chrysamin G (>2.5 μM) had also weak inhibitory effects whereas the apparent inhibitory effects of 17-AAG (>0.7 μM), riluzole (>25 μM), diclofenac sodium (>60 nM), sodium salicylate (>1250 μM) and Y-27632 (>12.5 μM) were due to toxic effects. C2-8 showed an inhibitory effect (>3 μM), however a concentration higher than 6 μM reduced the cell number. Chrysamine G, cystamine dihydrochloride, diclofenac, geldanamycin, sodiumsalyicylate, NDGA, creatin, coenzyme Q10 and threhalose showed no effects. The HDAC inhibitor scriptaid increased the aggregation of huntingtin (>25 μM), although the cell number decreased simultaneously.

Screening of a Natural Compound Library Identified Eleven New Modulators of Aggregation

To identify new compounds affecting huntingtin aggregation the inventors screened a natural compound library comprising 720 compounds (MicroSource Discovery Systems Inc., Gaylordsvile, Conn., USA).

Selection criteria for potential active compounds were set to a reduction of the FRET value higher than 30%, which equals ˜2 fold SD of the relative FRET value (1+/−0.17) and a maximal reduction of the cell number of 25% (FIG. 2D).

These conditions were fulfilled by 99 substances in the first screen. Of the 99 substances 37 substances were further selected based on their reduced toxicity and an efficacy in the range of 1-10 μM.

Subsequently, a second screening round was performed at concentration range 1, 3, and 10 μM, respectively. Dose response curves in the range of 0.7-50 μM and microscopy images confirm the results for 11 substances with the highest efficacy in a third screen. Quality of the assay system and screening procedure was assessed by the z′-factor (z′=0.71+/−0.14), a value for the data variability as well as the signal dynamic range. A z′-factor between 0.5-1 describes an “excellent assay” according to Zhang et al. (1999).

The 11 identified substances (S0 to S 10), showing highest effects in the aggregation process, share a high structural homology (>90%) based on a similarity search using CHED (ChemDB.com) (see Table 1). All compounds represent natural triterpenoids mainly isolated from Meliaceae spp. (6) and Khaya species (2). Three substances are derivatives of gedunin, three of khivorin and two of angolensic acid methylester.

The average EC₅₀ values are between 2-15 μM. Khayanthone and isogedunin revealed the strongest inhibitory activity with a EC₅₀ of 2 μM, followed by havanensin triacetat (3 μM) and 3-alphahydroxy-3-deoxy angolensic acid methylester (3 μM). The dose response curve and corresponding microscopy pictures for khayanthone, with an EC₅₀ (half-maximal inhibition) of 2 μM are diagrammed in FIG. 3.

Efficacy of the Eleven Natural Compounds in an In Vitro Filter Assay

To determine if the cellular environment is a prerequisite for the inhibitory effect of the isolated natural compounds or if the compounds directly interfere with the aggregation process, the inventors performed a cell free filter retardation assay (Scherzinger et al., 1997).

Briefly, GST-tagged mutant htt-exon-1 with 51 polyglutamines (GST-HDexQ51) is incubated with the compounds and factor Xa to remove the GST tag for proper aggregation (16 h, 23° C.). The aggregated peptides are denaturized by boiling in SDS buffer (10 min, 99° C.) and filtered though a cellulose acetate membrane. The insoluble aggregates are retained at the membrane and detected with an anti-huntingtin antibody.

The isolated natural compounds, PGL-135 and congo red were tested in a concentration range of 1-200 μM. Only congo red reliably inhibited the formation of aggregates (>50 μM). (Data not shown). Concentration of less than 50 μM congo red produced an enhancement of the aggregation process.

This indicates that the main effect of the compounds is not through direct interference with the formation of the amyloid-like huntingtin fibrils, but rather an effect coupled with a cellular context.

Khayanthone Has a Concentration-Dependent Effect on the Proteasome Activity.

Application of khayanthone to our cell models resulted in a decreased amount of huntingtin proteins pointing to a probably enhanced activity of the UPS caused by khayanthone. To confirm this we examined the chymotrypsin activity of the proteasome under the influence of khayanthone and compared data with known substances influencing the proteasomes' activity (gedunin, lactacystin, 17-AAG) (FIG. 4). Adminstration of the selected substances was performed for 24 h, and subsequently chymotrypsin activity of the UPS was determined by measuring the hydrolysis of a fluorogenic reporter (Suc-LLVY-AMC, Biomol).

Comparable to the HSP90 inhibitor 17-AAG, khayanthone enhances proteasome activity of about 40-50% in CHO-AA8/Tet-Off cells. The highest chymotrypsin activity (˜40%) was obtained at a concentration of 1 μM khayanthone whereas a decline in chymotrypsin activities (˜10%) was observed with increasing concentrations of khayanthone (3, 10 μM) (FIG. 4). Analysis characterizes khayanthone as a potent modulator of the proteasome activity.

Khayanthone Has an Effect on the Protein Concentration of HSP40, HSP70 and HSP90.

We examined effects of khayanthone on the expression of heat shock proteins, preferentially HSP40, HSP70 and HSP90, because of their impacts in neurodegenerative disease and cancer. Heat shock protein expression was determined in neuronal cells (SH-SY5Y cell line) by western blot analysis. Increase of khayanthone administration led to a slight decrease in heat shock protein expression of HSP40, HSP70, and HSP90, respectively (FIG. 5).

Khayanthone Improves Motor Activities in a Drosophila Model of HD

The most potent substance khayanthone was further tested in a HD Drosophila model which expresses mutant HDexQ93 selectively in neurons (Marsh et al., 2003).

Previous studies demonstrated that photoreceptors and motor activities are progressively affected in flies with age (Marsh et al., 2003).

Flies were mated at 25° C. in vials containing standard food supplemented with different concentrations of the compound tested. Adults were transferred to vials containing fresh food supplemented with different concentrations of khayanthone (100 μM, 250 μM, and DMSO control) every 3 days after eclosion.

Effects of khayanthone on the flies' motor ability were measured with the climbing assay. For this purpose flies were placed in a vial, then the height, which half of them were able to climb from the bottom within 60 sec, was determined. In addition, the time was measured which half of them needed to climb up 6 cm. The assays were repeated five times on each population sample.

Until day 18 there was no detectable difference between treated and untreated flies. This period is followed by a progressive loss of climbing activity started in DMSO fed flies (˜13% of the initial distance in 60 sec/4.4 times the initial time for a 6 cm distance). In comparison, khayanthone treated flies show less reduced motor ability (˜50% of the initial distance in 60 sec/2.3 times of the initial time for a 6 cm distance) (see FIG. 6).

The features disclosed in the foregoing description, in the claims and/or in the accompanying drawings may, both separately and in any combination thereof, be material for realizing the invention in diverse forms thereof.

### REFERENCES

Apostol, B. L., A. Kazantsev, S. Raffioni, K. files, J. Pallos, L. Bodai, N. Slepko, J. E. Bear, F. B. Gertler, S. Hersch, D. E. Housman, J. L. Marsh and L. M. Thompson (2003). A cell-based assay for aggregation inhibitors as therapeutics of polyglutamine-repeat disease and validation in Drosophila. Proc Natl Acad Sci USA 100: 5950-5955.

Arrasate, M., S. Mitra, E. S. Schweitzer, M. R. Segal and S. Finkbeiner (2004). Inclusion body formation reduces levels of mutant huntingtin and the risk of neuronal death. Nature 431: 805-810.

Bence, N. F., R. M. Sampat and R. R. Kopito (2001). Impairment of the ubiquitin-proteasome system by protein aggregation. Science 292: 1552-1555.

Bonelli, R. M. and P. Hofmann (2007). A systematic review of the treatment studies in Huntington's disease since 1990. Expert Opin Pharmacother 8: 141-153.

Cummings, C. J., Y. Sun, P. Opal, B. Antalffy, R. Mestril, H. T. Orr, W. H. Dillmann and H. Y. Zoghbi (2001). Over-expression of inducible HSP70 chaperone suppresses neuropathology and improves motor function in SCA1 mice. Hum Mol Genet 10: 1511-1518.

DiFiglia, M., E. Sapp, K. O. Chase, S. W. Davies, G. P. Bates, J. P. Vonsattel and N. Aronin (1997). Aggregation of huntingtin in neuronal intranuclear inclusions and dystrophic neurites in brain. Science 277: 1990-1993.

Ehrnhoefer D E, Bieschke J, Boeddrich A, Herbst M, Masino L, Lurz R, Engemann S, Pastore A, Wanker E E. EGCG redirects amyloidogenic polypeptides into unstructured, off-pathway oligomers. Nat Struct Mol Biol. 2008 June; 15(6):558-66.

Elangovan M, Day R N, Periasamy A (2002) Nanosecond fluorescence resonance energy transfer-fluorescence lifetime imaging microscopy to localize the protein interactions in a single living cell. J Microsc 205: 3-14

Heiser, V., S. Engemann, W. Brocker, I. Dunkel, A. Boeddrich, S. Waelter, E. Nordhoff, R. Lurz, N. Schugardt, S. Rautenberg, C. Herhaus, G. Barnickel, H. Bottcher, H. Lehrach and E. E. Wanker (2002). Identification of benzothiazoles as potential polyglutamine aggregation inhibitors of Huntington's disease by using an automated filter retardation assay. Proc Natl Acad Sci USA 99 Suppl 4: 16400-16406.

Holmberg, C. I., K. E. Staniszewski, K. N. Mensah, A. Matouschek and R. I. Morimoto (2004). Inefficient degradation of truncated polyglutamine proteins by the proteasome. Embo J 23: 4307-4318.

Igarashi S, Koide R, Shimohata T, Yamada M, Hayashi Y, Takano H, Date H, Oyake M, Sato T, Sato A, Egawa S, Ikeuchi T, Tanaka H, Nakano R, Tanaka K, Hozumi I, Inuzuka T, Takahashi H, Tsuji S (1998) Suppression of aggregate formation and apoptosis by transglutaminase inhibitors in cells expressing truncated DRPLA protein with an expanded polyglutamine stretch. Nat Genet 18: 111-117

Imarisio S, Carmichael J, Korolchuk V, Chen C W, Saiki S, Rose C, Krishna G, Davies J E, Ttofi E, Underwood B R, Rubinsztein D C. Huntington's disease: from pathology and genetics to potential therapies. Biochem J. 2008 Jun. 1; 412(2):191-209.

Karin N J. Cloning of transfected cells without cloning rings. Biotechniques. 1999 October; 27(4):681-2.

Lansbury, P. T. & Lashuel, H. A. A century-old debate on protein aggregation and neurodegeneration enters the clinic. Nature 443, 774-779 (2006).

Marsh, J. L., J. Pallos and L. M. Thompson (2003). Fly models of Huntington's disease. Hum Mol Genet 12 Spec No 2: R187-193.

Martin-Aparicio, E., A. Yamamoto, F. Hernandez, R. Hen, J. Avila and J. J. Lucas (2001). Proteasomal-dependent aggregate reversal and absence of cell death in a conditional mouse model of Huntington's disease. J Neurosci 21: 8772-8781.

Muchowski, P. J., G. Schaffar, A. Sittler, E. E. Wanker, M. K. Hayer-Hartl and F. U. Hartl (2000). Hsp70 and hsp40 chaperones can inhibit self-assembly of polyglutamine proteins into amyloid-like fibrils. Proc Natl Acad Sci USA 97: 7841-7846.

Novoselova, T. V., B. A. Margulis, S. S. Novoselov, A. M. Sapozhnikov, J. van der Spuy, M. E. Cheetham and I. V. Guzhova (2005). Treatment with extracellular HSP70/HSC70 protein can reduce polyglutamine toxicity and aggregation. J Neurochem 94: 597-606.

Piccioni F, Roman B R, Fischbeck K H, Taylor JP (2004) A screen for drugs that protect against the cytotoxicity of polyglutamine-expanded androgen receptor. Hum Mol Genet 13: 437-446

Pollitt S K, Pallos J, Shao J, Desai U A, Ma A A, Thompson L M, Marsh J L, Diamond M I (2003) A rapid cellular FRET assay of polyglutamine aggregation identifies a novel inhibitor. Neuron 40: 685-694

Rajan, R. S., M. E. Illing, N. F. Bence and R. R. Kopito (2001). Specificity in intracellular protein aggregation and inclusion body formation. Proc Natl Acad Sci USA 98: 13060-13065.

Rennel E, Gerwins P. How to make tetracycline-regulated transgene expression go on and off. Anal Biochem. 2002 Oct. 1; 309(1):79-84.

Ross C A, Poirier M A (2004) Protein aggregation and neurodegenerative disease. Nat Med 10 Suppl: S10-17

Sattler R, Charlton M P, Hafner M, Tymianski M (1997) Determination of the time course and extent of neurotoxicity at defined temperatures in cultured neurons using a modified multiwell plate fluorescence scanner. J Cereb Blood Flow Metab 17: 455-463

Scherzinger E, Sittler A, Schweiger K, Heiser V, Lurz R, Hasenbank R, Bates G P, Lehrach H, Wanker E E (1999) Self-assembly of polyglutamine-containing huntingtin fragments into amyloid-like fibrils: implications for Huntington's disease pathology. Proc Natl Acad Sci USA 96: 4604-4609

Sittler, A., R. Lurz, G. Lueder, J. Priller, H. Lehrach, M. K. Hayer-Hartl, F. U. Hartl and E. E. Wanker (2001). Geldanamycin activates a heat shock response and inhibits huntingtin aggregation in a cell culture model of Huntington's disease. Hum Mol Genet 10: 1307-1315.

Sittler A., Walter S., Wedemeyer N., Hasenbank R., Scherzinger E., Eickhoff H., Bates G., Lehrach H., Wanker E. SH3GL3 Associates with the Huntingtin Exon 1 Protein and Promotes the Formation of Polygln-Containing Protein Aggregates. Molecular Cell 2003, Volume 2, Issue 4, Pages 427-436

Takahashi T, Kikuchi S, Katada S, Nagai Y, Nishizawa M, Onodera O. Soluble polyglutamine oligomers formed prior to inclusion body formation are cytotoxic. Hum Mol Genet. 2008 Feb. 1; 17(3):345-56.

Waelter, S., A. Boeddrich, R. Lurz, E. Scherzinger, G. Lueder, H. Lehrach and E. E. Wanker (2001). Accumulation of mutant huntingtin fragments in aggresome-like inclusion bodies as a result of insufficient protein degradation. Mol Biol Cell 12: 1393-1407.

Wang W, Duan W, Igarashi S, Morita H, Nakamura M, Ross C A (2005b) Compounds blocking mutant huntingtin toxicity identified using a Huntington's disease neuronal cell model. Neurobiol Dis 20: 500-508

Warrick, J. M., H. L. Paulson, G. L. Gray-Board, Q. T. Bui, K. H. Fischbeck, R. N. Pittman and N. M. Bonini (1998). Expanded polyglutamine protein forms nuclear inclusions and causes neural degeneration in Drosophila. Cell 93: 939-949.

Wyttenbach, A., J. Carmichael, J. Swartz, R. A. Furlong, Y. Narain, J. Rankin and D. C. Rubinsztein (2000). Effects of heat shock, heat shock protein 40 (HDJ-2), and proteasome inhibition on protein aggregation in cellular models of Huntington's disease. Proc Natl Acad Sci USA 97: 2898-2903.

Zal T, Gascoigne N R (2004) Photobleaching-corrected FRET efficiency imaging of live cells. Biophys J 86: 3923-3939

Zal T, Zal M A, Gascoigne N R (2002) Inhibition of T cell receptor-coreceptor interactions by antagonist ligands visualized by live FRET imaging of the Thybridoma immunological synapse. Immunity 16: 521-534

Zhang J H, Chung T D, Oldenburg K R (1999) A Simple Statistical Parameter for Use in Evaluation and Validation of High Throughput Screening Assays. J Biomol Screen 4: 67-73

Zhang, X., D. L. Smith, A. B. Meriin, S. Engemann, D. E. Russel, M. Roark, S. L. Washington, M. M. Maxwell, J. L. Marsh, L. M. Thompson, E. E. Wanker, A. B. Young, D. E. Housman, G. P. Bates, M. Y. Sherman and A. G. Kazantsev (2005). A potent small molecule inhibits polyglutamine aggregation in Huntington's disease neurons and suppresses neurodegeneration in vivo. Proc Natl Acad Sci USA 102: 892-897.

1. A method for the treatment, diagnosis and/or prevention of a disease wherein said method comprises the use of a tetranortriterpernoid compound.

2. The method of claim 1, wherein the disease is a trinucleotide repeat disorder, amyloid disease, neurodegenerative disease, protein misfolding disease or tumor.

3. The method of claim 2, wherein the trinucleotide repeat disorder is a polyglutamine disease.

4. The method of claim 3, wherein the polyglutamine disease is selected from Huntington's disease (HD), dentatorubropallidoluysian atrophy (DRPLA), spinobulbar muscular atrophy (SBMA) and spinocerebellar ataxias (SCA).

5. The method, according to claim 1, used to reduce and/or inhibit the aggregation of amyloidogenic proteins.

6. The method of claim 5, wherein the aggregated protein is wildtype huntingtin or mutated huntingtin.

7. The method, according to claim 1, used to inhibit a heat shock proteins.

8. The method, according to claim 1, used to increase proteasome activity.

9. The method according to claim 1, wherein the tetranortriterpernoid compound is selected from the group, consisting of gedunin derivatives, khivorin derivatives, derivatives of angolensic acid methyl ester, angolensic acid, havanensin triacetate and khayanthone.

10. The method, according, to claim 1, wherein the tetranortriterpernoid compound is selected from the group consisting of;
havanensin triacetate (S0),
khayanthone (S1),
angolensic acid methylester
3-alphahydroxy-3-deoxy angolensic acid methylester (S3),
isogedunin (S4),
epoxy (1,2 alpha) 7-deacetocy-7-oxo-deoxydihydrogedunin (S5),
1,3-dideacetyl khivorin (S6),
deacetoxy-7-oxisogedunin (57),
1,7 -dideacetoxy-1,7-dioxokhivorin (S8),
3-beta-acetoxydeocyangoensic acid methylester (S9),
1,3-dideacetyl-7-deacetoxy-7-oxokhivorin (S10),
and salts and derivatives thereof.

11. The method, according to claim 1, wherein the tetranortriterpernoid is khayanthone (S1).

12. (canceled)

13. A pharmaceutical composition, comprising one or more tetranortriterpernoids, selected from the group consisting of:
havanensin triacetate (SO),
khayanthone (S1),
angolensic acid methylester (S2)
3-alphahydroxy-3-deoxy angolensic acid methylester (S3),
isogedunin (S4),
epoxy (1,2 alpha) 7-deacetocy-7-oxo-deoxydihydrogedunin (S5),
1,3-dideacetyl khivorin (S6),
deacetoxy-7-oxisogedunin (S7),
1,7 -dideacetoxy-1,7-dioxokhivorin (S8),
3-beta-acetoxydeocyangoensic acid methylester (S9),
1,3-dideacetyl-7-deacetoxy-7-oxokhivorin (S10),
and pharmaceutically acceptable salts and derivatives thereof, and optionally, pharmaceutically acceptable excipients and/or carriers.

14. (canceled)

15. A nucleic acid, comprising the nucleotide sequences of two huntingtin fragments, wherein at least one is selected from huntingtin exon 1 (HDex1, wildtype) or huntingtin N-terminal fragment of amino acids 1-514 (HD514, wildtype).

16. The nucleic acid of claim 15 in the form of a bidirectional construct.

17. The nucleic acid of claim 15, wherein each of the two huntingtin fragments is a fusion protein with a chromophor.

18. The nucleic acid of claim 17, wherein the chromophor is green fluorescent protein (GFP) or a derivate of GFP or enhanced green fluorescent protein (EGFP).

19. The nucleic acid of claim 17, wherein the chromophor is N-terminal or C-terminal fused to each of the two huntingtin fragments.

20. The nucleic acid of claim 18, comprising HDex1Q17-YFP and HDex1Q17-CFP, HDex1Q68-YFP and HDex1Q68-CFP. HD514Q17-YFP and HD514Q17-CFP, or HD514Q68-YFP and HD514Q68-CFP, or comprising at least HDex1Q68-YFP.

21. The nucleic acid of claim 15, further comprising a Tet-regulated promoter.

22. The nucleic acid of claim 21, wherein the Tet-regulated promoter comprises a tetracyclin responsive clement (TRE) and CMV promoter(s).

23. A cell, comprising a nucleic acid of claim 15.

24. The cell of claim 23, which is a cell of a Tet-off cell line.

25. An in vitro method for assessing the aggregation of huntingtin in mammalian cells, comprising the steps of: a) providing one or more nucleic acids, which comprise the nucleotide sequences coding for two huntingtin fragments, b) transfecting the nucleic, acid(s) into mammalian cells, c) co-expressing the two huntingtin fragments in the transfected mammalian cells, and d) detecting the aggregation of the two huntingtin fragments.

26. The method of claim 25, wherein the huntingtin fragments are selected from huntingtin exon 1 (amino acids 1-90) (HDex1) and huntingtin N-terminal fragment of amino acids 1-514(HD514).

27. The method of claim 25, wherein the huntingtin fragments comprise a polyglutamine sequence (polyQ sequence).

28. The method of claim 27, wherein huntingtin fragments comprise the same polyQ sequence.

29. The method of claim 25, wherein each of two huntingtin fragments is a fusion protein with a chromophor.

30. The method of claim 29, wherein the chromphor is green fluorescent protein (GFP) or a derivate of GFP or enhanced green fluorescent protein (EGFP).

31. (canceled)

32. The method of claim 25, wherein the nucleic acid in step a) is a nucleic acid, comprising the nucleotide sequences of two huntingtin fragments, wherein at least one selected from huntingtin exon 1 (HDex1, wildtype) or huntingtin N-terminal fragment of amino acids 1-514 (HD514, wildtype)

33. The method of claim 25, wherein the detection of the aggregation in step d) is carried out by measuring the fluorescence resonance energy transfer (FRET) signal.

34. (canceled)

35. A method for the identification of a compound, which modulates the aggregation of huntingtin, comprising a method of claim 25 and further contacting the compound with the transfected mammalian cell which co-expresses the two huntingtin fragments.

36. A kit for assessing the aggregation of huntingtin, comprising a nucleic acid of claim 15 and optionally a cell comprising a nucleic acid of claim 15.


SEQUENCE LISTING (7 sequences)

SEQ ID NO: 1 - protein, 3144 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 2 - protein, 84 aa, Homo sapiens
MATLEKLMKAFESLKSFQQQQQQQQQQQQQQQQQPPPPPPPPPPPQLPQPPPQAQPLLPQPQPPPPPPPPPPGPAVAEEPLHRP

SEQ ID NO: 3 - protein, 135 aa, Artificial Sequence
  Huntingtin Exon 1 with 68 Q (Glu) repeats
MATLEKLMKAFESLKSFQQQQQQQQQQQQQQQQQQQQQQQQQQQQQQQQQQQQQQQQQQQQQQQQQQQQQQQQQQQQQQQQQQQQPPPPPPPPPPPQLPQPPPQAQPLLPQPQPPPPPPPPPPGPAVAEEPLHRP

SEQ ID NO: 4 - DNA, 29 bp, Artificial Sequence
  Primer
cgcgaattccatggcgaccctggaaaagc

SEQ ID NO: 5 - DNA, 31 bp, Artificial Sequence
  Primer
cgcggatcctttggtcggtgcagcggctcct

SEQ ID NO: 6 - DNA, 29 bp, Artificial Sequence
  Primer
cgcgaattccatggcgaccctggaaaagc

SEQ ID NO: 7 - DNA, 32 bp, Artificial Sequence
  Primer
cgcggatcctcatggtcggtgcagcggctcct